(19) United States
(12) Patent Application Publication
(10) Pub. No.: US 2010/0102142 A1
(43) Pub. Date: Apr. 29, 2010

(54) SCENT DISPENSER ASSEMBLY

(76) Inventor: Daria Tagliareni, Spring Lake, NJ (US)

Correspondence Address: RAYMOND R. MOSER JR., ESQ.;MOSER IP LAW GROUP, 1030 BROAD STREET, 2ND FLOOR, SHREWSBURY, NJ, 07702, US

(21) Appl. No.: 12/607,771

(22) Filed: Oct. 28, 2009

Related U.S. Application Data
(60) Provisional application No. 61/108,947, filed on Oct. 28, 2008.

(51) Int. Cl. A61L 9/04

(52) U.S. Cl. 239/36

(57) ABSTRACT

Scent dispenser assemblies for providing an extended time-release of a desired scent are disclosed herein. In some embodiments, a scent dispenser assembly may include a substrate configured to release a desired scent over a period of time; and a housing for retaining the substrate, the housing configured to be worn by a user. In some embodiments, a scent dispensing system may include a plurality of substrates each configured to release a desired scent over a period of time and having a three dimensional shape, wherein the three dimensional shape is at least one of a sphere or a polyhedron; and a housing for retaining the plurality of scent dispensers.

### CROSS REFERENCE TO RELATED APPLICATIONS

This application claims benefit of U.S. provisional application Ser. No. 61/108,947, filed Oct. 28, 2008, which is herein incorporated by reference.

### BACKGROUND

1. Field

Embodiments of the present invention generally relate to the field of dispensers for scents, such as perfume.

2. Description of the Related Art

After nickel, fragrances are the second most common contact allergens. Contact allergens are substances that trigger allergic skin reactions in some people when they come in contact with the skin. Contact allergies to fragrances are increasing worldwide. Perfumes are typically placed on the skin or clothing where they are free to interact with the skin surface.

Therefore, there exists a need for an improved perfume dispensing system for personal scent markets.

### SUMMARY

Scent dispenser assemblies for providing an extended time-release of a desired scent are disclosed herein. In some embodiments, a scent dispenser assembly may include a substrate configured to release a desired scent over a period of time; and a housing for retaining the substrate, the housing configured to be worn by a user. In some embodiments, a scent dispensing system may include a plurality of substrates each configured to release a desired scent over a period of time and having a three dimensional shape, wherein the three dimensional shape is at least one of a sphere or a polyhedron; and a housing for retaining the plurality of scent dispensers.

In some embodiments, dispenser assemblies are provided for housing disposable scent dispensers, in the form of solid substrates that may be microencapsulated with a desired scent, or porous substrates that may be pre-scented and/or have a desired scent applied thereto. In some embodiments, designed jewelry pieces may act as a dispenser/assembly housing for these scent dispensers. The solid substrate scent dispensers are made from cellulose acetate designed to encapsulate scents and to slowly release such scents over time. Non-scented porous substrates may act as a scent receptacle, where a desired scent, such as a perfume, may be added to the porous substrate by a consumer as desired.

In some embodiments, the dispenser assembly housing may include an aperture for insertion or removal of the scent dispenser from the exterior of or an orifice of the dispenser assembly. In some embodiments, a scent dispenser in the form of a disposable preloaded microencapsulated scented substrate is configured to be inserted into the aperture and locked within the jewelry orifice (as discussed below with respect to FIG. 4). In some embodiments, the stylized jewelry dispenser engages an aperture of the dispenser assembly to selectively secure a pre-scented solid substrate unit to the jewelry piece (as discussed below with respect to FIG. 2). In some embodiments, the solid substrate disposable preloaded microencapsulated scent is molded into a stone shape that snap-fits into the aperture (as discussed below with respect to FIGS. 1 and 3). In some embodiments, the scent dispenser may be in the form of a solid or porous substrate that may be pre-formed in a desired shape. One or more of the scent dispensers may be disposed in a receptacle for housing the scent dispensers (as discussed below with respect to FIGS. 5 and 6).

Other variations and embodiments are disclosed below with respect to the Detailed Description.

### BRIEF DESCRIPTION OF THE DRAWINGS

The accompanying drawings, schematically illustrate embodiments of the present invention and together with the general description given above and the detailed description given below serve to explain the principles of the invention. The Figures herein are illustrative only and are not drawn to scale and may be simplified for clarity.

FIG. 1 is a side perspective view of a scent dispenser assembly in accordance with some embodiments of the present invention;

FIG. 2 is a side perspective view of a scent dispenser assembly in accordance with some embodiments of the present invention;

FIG. 3 is a front view of a scent dispenser assembly in accordance with some embodiments of the present invention;

FIG. 4 depicts front and back views of a scent dispenser assembly in accordance with some embodiments of the present invention;

FIG. 5 is a side view of a scent dispenser assembly along with a top view of scent dispensers in accordance with some embodiments of the present invention; and

FIG. 6 is a side view of a scent dispenser assembly along with a top view of scent dispensers in accordance with some embodiments of the present invention.

FIG. 7 is a side view of a scent dispenser assembly in accordance with some embodiments of the present invention.

It is to be noted, however, that the appended drawings illustrate only typical embodiments of this invention and are therefore not to be considered limiting of its scope, for the invention may admit to other equally effective embodiments. It is also contemplated that features of one embodiment may be beneficially incorporated in other embodiments, including those not explicitly described, without a specific description of the combined embodiment.

### DETAILED DESCRIPTION

Embodiments of a scent dispenser assembly are provided herein for retaining a replaceable or replenishable scent dispenser within a housing or receptacle. The housing for the scent dispenser may have a variety of forms, as discussed below, which enable the scent dispenser assembly to be used a variety of arrangements. In some embodiments, the scent dispenser assembly may be used as consumer jewelry (e.g., necklaces, anklets, belly chains, belts, bracelets, cuffs, earrings, rings, piercings, pins, brooches, or the like) which allows for a time-extended release of a non-contact scent (i.e., a scent that does not come in contact with the skin of a user). In some embodiments, the housing or receptacle may have a form, such as a bowl, a hollow vessel or container, or the like, and may be utilized to hold the scent dispensers, for example, for home or office use (e.g., in closets or drawers, on tabletops, or the like). Generally a scope of scented formulations may include but is not limited to standard perfumes, aromatherapy oils, and the like.

As described in more detail below, the scent dispenser may be formed from a solid or porous substrate having a “thin” form factor (e.g., a substrate having a thickness that is much smaller that the length, width, or other dimensions that define the primary surface area of the substrate) or a three-dimensional form factor (e.g., a sphere, a polyhedron, and the like). The scent dispenser may be pre-scented or may comprise non-scented filtration pads (e.g., porous pads, or a porous substrate) readied to receive and retain consumer provided scents. The scent dispenser generally provides scent dispersion over extended periods as compared to the average rate of evaporation of a scent disposed on a surface (such as clothing or skin). For example in some embodiments the scent dispenser may provide scent dispersion for a 30 day period or longer. In some embodiments, the scent dispensers may encapsulate the scent or retain the scent in a reservoir and can retain and emit a scent for an extended period of time, in some embodiments up to 5 years, and in some cases even longer, depending upon the scent formulation. The scent dispenser may be coupled to or placed into an orifice of a housing or receptacle, where chemical components found in the typical perfume formulation that elicit an allergic response do not come into contact with a user's skin.

The scent dispenser generally may have any suitable form factor for use in connection with the housing for retaining the scent dispenser as described below. In some embodiments, the scent dispenser may be in the form of a molded or otherwise formed three-dimensional shape, such as a sphere, a polyhedron, or the like. In some embodiments, the three-dimensional shape may be a polyhedron that emulates a cut gemstone, or other suitable three-dimensional shape as desired. In some embodiments, the scent dispenser substrate may have a thin form factor, such as a strip, pad, or the like.

The scent dispenser may be formed from a scented solid substrate, such as a polymer, cellulose acetate, or the like. The solid substrate may be scented by microencapsulation of a desired scent during fabrication of the substrate or underlying substrate material. For example, scented cellulose acetate may be made from processed wood pulp processed using acetic anhydride to form acetate flake, from which products are made into thin substrates or other shapes, such as the three-dimensional form factors discussed herein, using microencapsulating technology for scent retention and release. In some embodiments, a scented cellulose acetate scent dispenser may be formulated as discussed in United States Patent Application Publication No. 2006-0104933, published May 18, 2006, entitled, “Process for Preparing Scented Cellulosics and Products Prepared Thereby,” and assigned to ROTUBA EXTRUDERS, INC, of Linden, N.J. In some embodiments, the scent dispenser may be colored for aesthetics.

The scent dispenser may alternatively be formed from a porous substrate, such as bonded fiber components (e.g., bonded fibers of cellulose acetate), polyurethane foam, and the like, comprising one or more reservoirs for retaining a scented formulation (e.g., a liquid, powder, gas, or the like). The bonded fiber components may be formed in a thin form factor or in a three dimensional form, and may be constructed to handle or manipulate liquids or vapors at the microliter or milliliter scale. In some embodiments, the porous substrate may be pre-scented. Alternatively, the porous substrate may initially be un-scented and adapted for receiving, retaining, and dispersing an applied scent. The porous substrate may be replenishable, i.e., the scent dispenser may receive, retain, and disperse additional applied scent.

In some embodiments, the porous substrate may comprise a matrix of bonded cellulous acetate fibers for containing and controlling delivery of scented chemistry formulations, perfume concentrates, color pigments, and mixtures, and/or other odor absorbent technology. The bonded cellulous acetate fiber substrate enables control of the flow rates of liquids, such as perfumes, within the scented substrate. Scented fluid transfer or flow can be modified or enhanced with liquid treatment or conversion of the component, via impregnation or overlay of functional chemical modifiers, to selectively add, remove, or otherwise manipulate properties of the liquid. Wicking rates and volumes may be selected to work with individual scented chemical formulation.

Additionally, the matrix of bonded cellulous acetate fibers may provide visual identification or visual functionality via the use of color. Engineered colored components may be added to the substrate for one or more of aesthetic purposes, to signify a change in state of a scent dispenser (e.g., from wet to dry, from full to empty), to indicate an amount of scent remaining in a scent dispenser, and/or to indicate an end of life for a scent dispenser (e.g., no remaining scent and thus time to replace/replenish the scent dispenser). Changes in state and/or available scent level in a scent dispenser may be indicated, for example, by different colors and/or by a change in color intensity.

In some embodiments, the scent dispenser may be formed from a polyurethane foam that allows intimate impregnation of scented powders, liquids and solids into the resultant foam products during manufacture of the scent dispenser. The polyurethane foam may comprise a variety of cell sizes, textures, and densities. The manufacturing process for the polyurethane foam scent dispensers allows for the production of extremely clean foam with extremely low amounts of extractables or residuals while achieving a desired scent distribution. The foam may take the form of a thin substrate or may have a three-dimensional shape. In some embodiments, the foam may be hardened, cut, and polished to provide a desired look and feel to the scent dispenser.

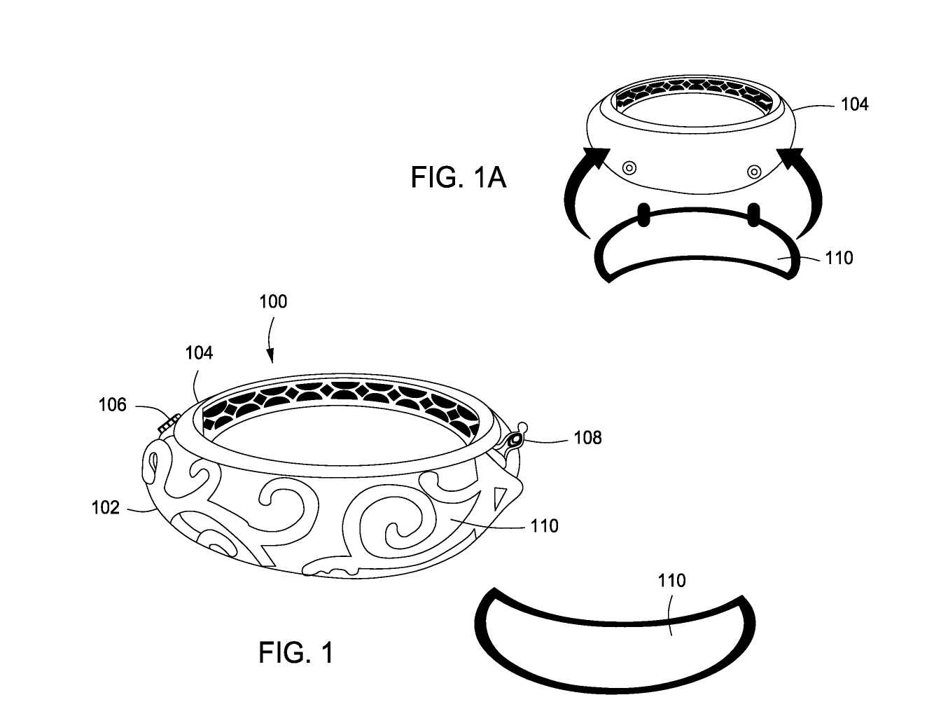

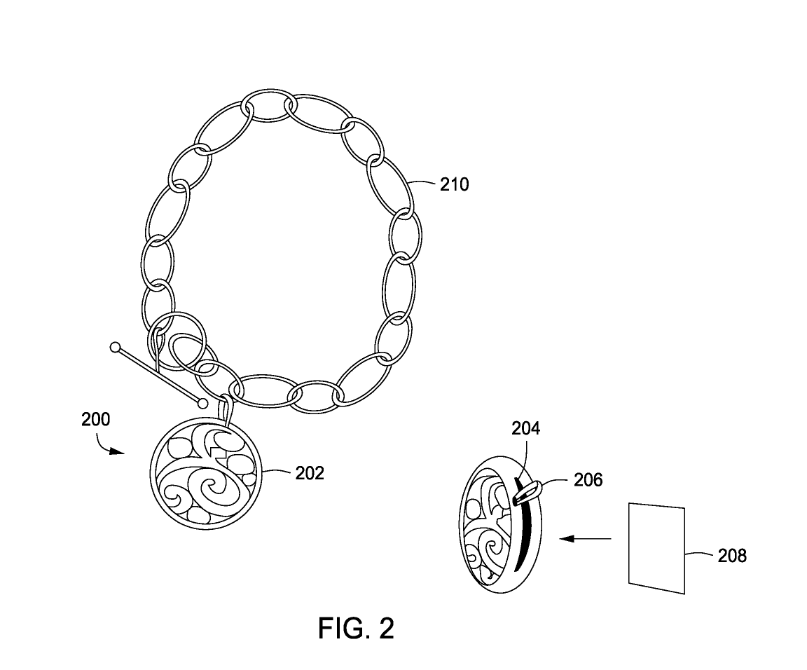

The housing may be fabricated from any suitable material, such as plastic, polymers, metal, glass, wood, combinations thereof, or the like, and may be transparent, semi-transparent, opaque, or combinations thereof. The housing may have any suitable shape, such as a round, square, flower, or other motifs, and may have vented areas on one or more outer surfaces that are fluidly coupled with the scent dispenser to allow the scent from the scent dispenser to escape. In some embodiments, the housing may include an aperture having, in some embodiments, a length of about 0.50 inches and a thickness of about 0.090 inches formed therein, although other suitable sizes may be utilized. Such an aperture facilitates access to the interior volume of housing for accessing a scent dispenser retained within the housing. The housing may additionally include one or more apertures to allow a fragrance to be dispersed from the dispenser assembly (such as apertures depicted in FIGS. 1 and 2 described below, or the like). In some embodiments, the housing may retain more than one scent dispenser.

As discussed in further detail with respect to FIGS. 1-6, below, in some embodiments, the scent dispenser may be in the form of a disposable solid substrate preloaded microencapsulated molded shape configured to engage with the scent dispenser assembly housing in a manner that retains the scent dispenser in the dispenser assembly. In some embodiments, the scent dispenser may be in the form of a disposable solid substrate preloaded microencapsulated shape and may be accessed for insertion into and removal from the scent dispenser assembly housing. This design retains the scent dispenser in an aperture of the scent dispenser assembly housing to selectively secure the scent dispenser in place in the dispenser assembly housing. In some embodiments, the scent dispenser may be in the form of a solid substrate pre-scented disposable preloaded microencapsulated molded shape that may be snap-fit into an aperture of the dispenser assembly housing. In some embodiments, the scent dispenser may be in the form of a non-scented porous substrate, adapted to receive, retain, and disperse an applied scent, that may be inserted into an aperture of the dispenser assembly housing.

FIG. 1 is a side perspective view of a scent dispenser assembly 100 in accordance with some embodiments of the present invention. In some embodiments, such as depicted in FIG. 1, the dispenser assembly 100 may comprise a cover 102 coupled to a housing 104 by a hinge 106. Although the hinge 106 is shown as a separate element coupling the cover 102 to the housing 104, the hinge 106 may be an integral part of the cover 102 and housing 104, for example, as a living hinge in embodiments where the housing 104 and cover 102 are molded as a single component. When the dispenser assembly 100 is closed, a latch 108 or other suitable mechanism secures the cover 102 to the housing 104. In some embodiments, the cover 102 may be secured to the housing 104 in alternative manners, for example, by mating geometry, snap fit, bayonet configuration, threaded engagement, removable adhesive, fasteners, clips, springs or other suitable arrangement for retaining the cover 102 to the housing 104.

A scent dispenser 110 is retained in the dispenser assembly 100 between the cover 102 and the housing 104. The scent dispenser 110 has a three-dimensional form factor adapted for fitting within and/or being coupled to the housing 104. As previously described, the scent dispenser 110 may be formed from a scented solid substrate or a porous substrate adapted for receiving, retaining, and dispersing a scent. In some embodiments, the scent dispenser 110 may be pigmented or colored. For example, the scent dispenser 110 may be colored in a random or abstract manner, or to emulate a gemstone, a precious stone, or the like.

In some embodiments, for example as depicted in FIG. 1A, the scent dispenser 110 may be coupled directly to the housing 104 without any need for the cover 102, such as by snap-fit (as depicted), or other suitable means, such as by adhesive, clips, fasteners, or the like. In such embodiments, the cover 102 may be provided for aesthetics or may not be used.

FIG. 2 depicts is a side perspective view of a scent dispenser assembly 200 in accordance with some embodiments of the present invention. The scent dispenser assembly 200 includes a body 202 (i.e., a housing) coupled to a holder 210. The body 202 includes an orifice or receptacle 204 and a safety closure 206, which together define an interior volume adapted to retain a replaceable scent dispenser 208 therein. The scent dispenser 208 may be formed from a scented solid or a porous substrate, as previously described, and may have a thin form factor, such as a pad, strip, or the like, adapted to fit within and be removable from the body 202. The holder 210 may illustratively comprise a flexible element, such as a chain, rope, wire, cord, string, or other flexible form, such as may be utilized as a bracelet, a necklace, an anklet, a belly chain, a belt, or the like. In some embodiments, the holder 210 may comprise a rigid element to facilitate wearing the scent dispenser assembly as a piercing, a pin, a brooch, a barrette, or the like. The body 202 may be selectively separated from the holder 210 to facilitate providing different style/color bodies 202 in the dispenser assembly 200.

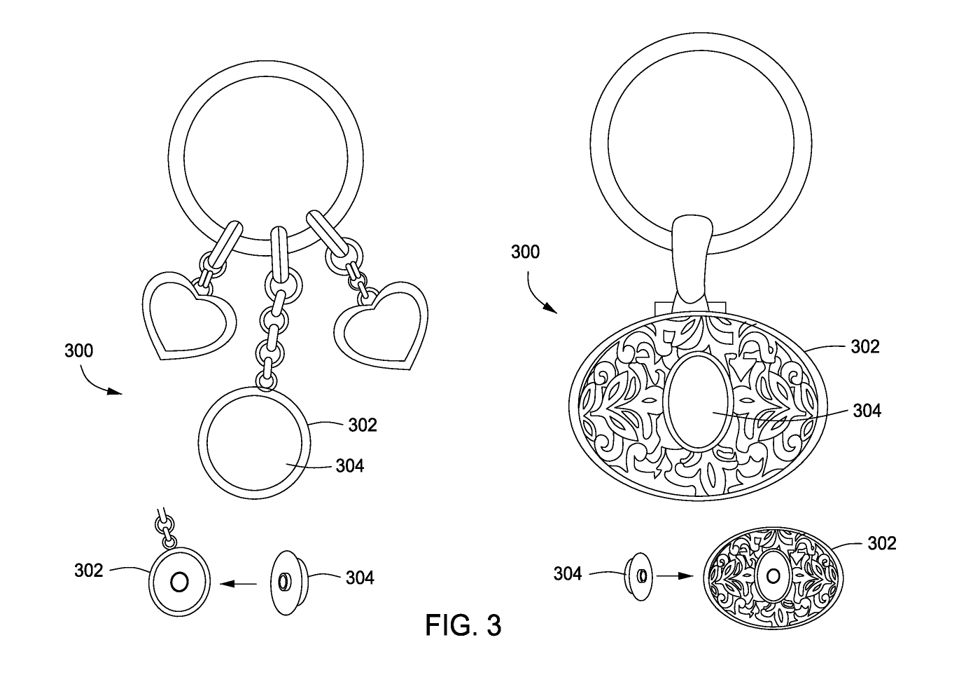

FIG. 3 is a front view of a scent dispenser assembly 300 in accordance with some embodiments of the present invention. FIG. 3 depicts an embodiment the present invention where the dispenser assemblies 300 are configured to be attached to a dangling charm such as, for example, a keychain, a bracelet, a necklace, or the like. The dispenser assemblies 300 each comprise a housing 302 and a scent dispenser 304. The scent dispensers 304 each have a three-dimensional form factor, such as a decorative form factor in the shape of a heart, a circle, or the like, adapted for coupling to a suitable housing 302. The scent dispensers 304 are illustratively configured with a mounting prong that mates with a corresponding feature formed in the housing 302 for coupling each scent dispenser 304 to a corresponding housing 302.

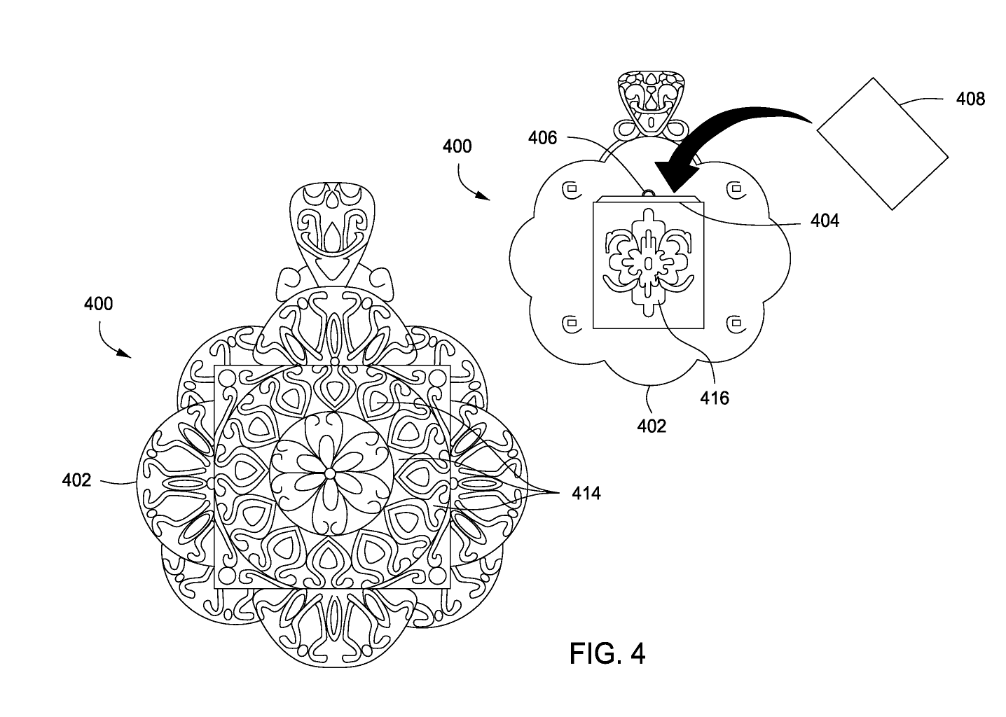

FIG. 4 depicts front and back views of a scent dispenser assembly 400 in accordance with one or more embodiments of the present invention. The dispenser assembly 400 comprises a housing 402 that includes an orifice or receptacle 404 and, optionally, a safety closure 406, which together define an interior volume adapted to retain a replaceable scent dispenser 408 therein. The scent dispenser 408 may have a thin form factor, such as a strip or pad, adapted for being inserted into and removed from the receptacle 404. The housing 402 further includes one or more openings 414 on the front of the housing 402 to facilitate allowing the scent from the scent dispenser 408 to escape from the receptacle 404 of the housing 402 through the openings 414. In some embodiments, housing 402 may further include one or more openings 416 on the back of the housing 402 to facilitate allowing the scent from the scent dispenser 408 to escape from the receptacle 404 of the housing 402 through the openings 416. The dispenser assembly 400 may be configured as a charm or other design to be worn, for example, as a necklace or bracelet, although other configurations are contemplated.

The dispenser assembly 400 is configured to facilitate an attachment to a holder, loop, or linked chain, such as a bracelet necklace chain, or the like. In some embodiments, the housing 402 may comprise a recess or channel to accommodate a scent dispenser in the form of a thin strip, pad, or similar thin form factor. Additionally or alternatively, the housing 402 may comprise a mounting prong for a scent dispenser in the form of a scented substrate in the form of a stone to snap into the surface of the housing 402. (See, for example FIGS. 1 and 3).

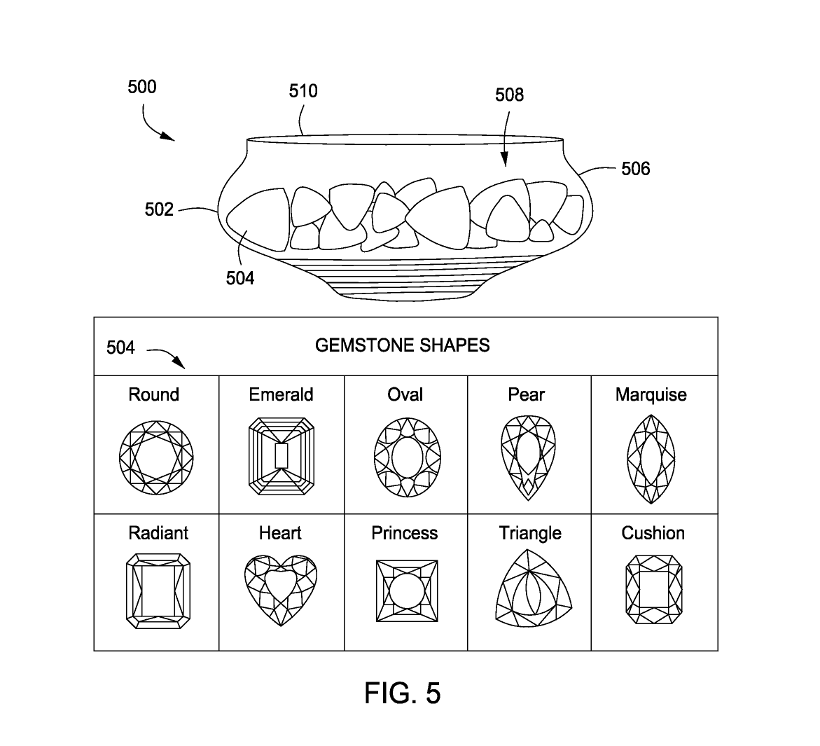

FIG. 5 is a side view of a scent dispenser assembly 500 along with a top view of scent dispensers in accordance with some embodiments of the present invention. In some embodiments, and as depicted in FIG. 5, the dispenser assembly 500 may be comprise a receptacle 502 for retaining one or more scent dispensers 504 therein. The receptacle 502 may take any suitable form and typically includes a body 506 having an inner volume 508 for holding the one or more scent dispensers 504; additionally, the receptacle 502 comprises one or more openings 510 to provide access to the inner body to insert and/or remove the one or more scent dispensers 504 and to allow the scent from the one or more scent dispensers 504 to escape from the receptacle 502. Examples of suitable forms for the receptacle 502 include, but are not limited to, bowls (as shown), vases, or the like.

The scent dispensers 504 may be formed into a desired three-dimensional shape, such as a cut jewel (as shown), an amorphous stone, or other geometric shapes (such as round, square, rectangular, triangular, polygonal, oval, random, or the like, or combinations thereof). For example, as depicted in FIG. 5, the receptacle 502 may comprise a glass bowl for retaining a plurality of scent dispensers 504 that may have the form of different styles of cut jewels in similar or different colors. An end user can arrange the scent dispensers 504 as desired to have common color or shape or to have a mixture of various colors and/or shapes.

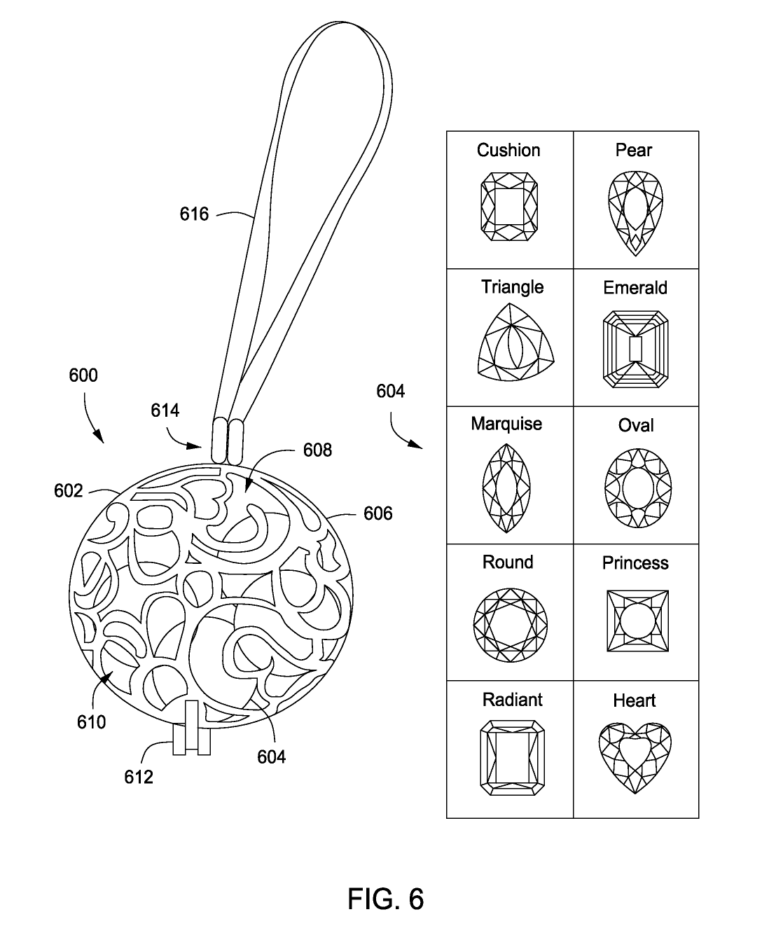

FIG. 6 is a side view of a scent dispenser assembly 600 along with a top view of scent dispensers in accordance with some embodiments of the present invention. As depicted in FIG. 6, the dispenser assembly 600 may comprise a housing, or receptacle 602 for retaining one or more scent dispensers 604 therein. The receptacle 602 may take any suitable form and typically includes a body 606 having an inner volume 608 for holding the one or more scent dispensers 604 and one or more openings 610 to allow the scent from the one or more scent dispensers 604 to escape from the receptacle 602. The receptacle 602 may further have an opening mechanism to provide access to the inner body to insert and/or remove the one or more scent dispensers 604 therefrom. For example, as depicted in FIG. 6, the receptacle 602 may have a clamshell form with two halves coupled together at a hinge 612 to facilitate opening and closing the receptacle 602 by moving the halves with respect to each other. A clasp or other locking mechanism 614 may be provided for retaining the receptacle 602 in the closed position. In some embodiments, a string, ribbon, rope, belt, or other flexible material may be provided to form a loop 616 (e.g., a holder) extending from the receptacle 602 (in some embodiments, from the locking mechanism 614 thereof), to facilitate hanging the receptacle 602 from a desired location, such as from a closet rod, a curtain rod, a nail or other protrusion from a wall, or the like. The scent dispensers 604 may be formed into a desired shape, as described above with respect to scent dispensers 504.

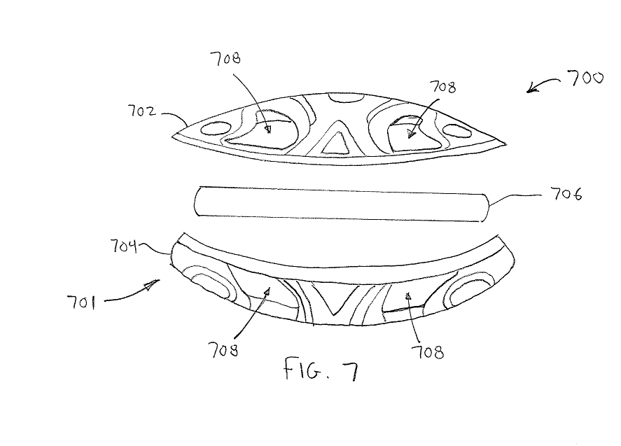

FIG. 7 is a side view of a scent dispenser assembly 700 in accordance with some embodiments of the present invention. As depicted in FIG. 7, the dispenser assembly 700 may comprise a housing or receptacle 701 for retaining one or more scent dispensers 706 therein. The receptacle 701 may take any suitable form and typically includes a body 704 having an inner volume for holding the one or more scent dispensers 706 and one or more openings 708 to allow the scent from the one or more scent dispensers 706 to escape from the receptacle 701. The receptacle 701 may further have a lid or top 702 that interfaces with the body 704 in a locking or non-locking fashion to cover the one or more scent dispensers 706. The lid 702 may also have one or more openings 708 disposed therein to allow the scent from the one or more scent dispensers 706 to escape from the receptacle 701. A single scent dispenser 706 formed in the shape of a plate is shown in FIG. 7. However, one or more scent dispensers 706 may be provided and may be formed into any desired shape, as described above with respect to scent dispensers 504.

Thus, scent dispenser assemblies suitable for retaining a replaceable and/or replenishable scent dispenser for dispensing consumer product have been provided. The scent dispenser assembly advantageously facilitates dispensing a scent while facilitating ease of replacement of a scent dispenser retained within and/or coupled to the dispenser assembly housing.

While the foregoing is directed to embodiments of the present invention, other and further embodiments of the invention may be devised without departing from the basic scope thereof.

1. A scent dispenser assembly, comprising:
a substrate configured to release a desired scent over a period of time; and
a housing for retaining the substrate, the housing configured to be worn by a user.

2. The assembly of claim 1, wherein the substrate is removably disposed in a receptacle of the housing.

3. The assembly of claim 1, wherein the substrate is removably retained within a receptacle of the housing by a closure mechanism.

4. The assembly of claim 1, wherein the substrate is a solid polymer material having a preloaded microencapsulated scent.

5. The assembly of claim 1, wherein the substrate is porous and adapted to receive and disperse a scent applied to the porous substrate.

6. The assembly of claim 1, wherein the substrate comprises at least one of cellulose acetate, a matrix of bonded cellulous acetate fibers, or polyurethane foam.

7. The assembly of claim 1, wherein the substrate comprises a polyurethane foam having a desired scent impregnated within the foam.

8. The assembly of claim 1, wherein the substrate changes color to visually indicate a level of remaining scent.

9. The assembly of claim 1, wherein the substrate has a thin form factor and wherein the housing further comprises a slot to removably receive the substrate therein.

10. The assembly of claim 1, wherein the substrate comprises a three dimensional shape and wherein the housing further comprises an aperture or a recess to removably receive the substrate.

11. The assembly of claim 10, wherein the substrate and the housing each further comprise one or more mating features to removably couple the substrate and the housing.

12. The assembly of claim 10, wherein the housing further comprises a permeable cover coupled to the housing via a hinge to selectively enclose and retain the substrate within the aperture of the housing.

13. The assembly of claim 1, wherein the housing further comprises a connector coupled to at least one of a flexible element having a clasp to selectively couple the flexible element to the housing or to another flexible element coupled to the housing, or a rigid element configured to allow the scent dispenser assembly to be worn as a piercing.

14. The assembly of claim 1, wherein the housing is configured as one of a necklace, a bracelet, a cuff, an anklet, a belly chain, a ring, an earring, a brooch, a pin, a barrette, or a belt.

15. A scent dispensing system, comprising:
a plurality of substrates each configured to release a desired scent over a period of time and having a three dimensional shape, wherein the three dimensional shape is at least one of a sphere or a polyhedron; and
a housing for retaining the plurality of scent dispensers.

16. The system of claim 15, wherein at least one substrate is a solid polymer having a preloaded microencapsulated scent.

17. The system of claim 15, wherein at least one substrate comprises a porous material adapted to receive and disperse a scent applied thereto.

18. The system of claim 15, wherein at least one substrate comprises at least one of cellulose acetate, a matrix of bonded cellulous acetate fibers, or polyurethane foam.

19. The system of claim 15, wherein at least one substrate changes color to visually indicate a level of remaining scent.

20. The system of claim 15, wherein at least one substrate comprises a polyurethane foam having hardened and polished surfaces.

21. The system of claim 15, wherein at least one substrate comprises a polyurethane foam having a desired scent impregnated within the foam.

22. The system of claim 15, further comprising a holder, extending from the housing, for enabling the housing to be hung.

23. The system of claim 15, wherein the three dimensional shape is a polyhedron shaped to emulate a cut gemstone.

24. The system of claim 15, wherein the holder is configured as an open container.

25. The system of claim 15, wherein the holder further comprises a recess for receiving the plurality of substrates and a lid to cover the recess and retain the plurality of substrates within the holder.
